(19) United States
(12) Patent Application Publication
(10) Pub. No.: US 2002/0119198 A1
(43) Pub. Date: Aug. 29, 2002

(54) Self-emulsifying drug delivery systems for extremely water-insoluble, lipophilic drugs

(76) Inventors: Ping Gao, Portage, MI (US); Walter Morozowich, Kalamazoo, MI (US); Narmada Shenoy, Sunnyvale, CA (US)

Correspondence Address: MARSHALL, GERSTEIN & BORUN, 6300 SEARS TOWER, 233 SOUTH WACKER, CHICAGO, IL, 60606-6357, US

(21) Appl. No.: 09/909,691

(22) Filed: Jul. 20, 2001

Related U.S. Application Data
(60) Provisional application No. 60/220,376, filed on Jul. 24, 2000.

(51) Int. Cl. A61K 9/14

(52) U.S. Cl. 424/486

(57) ABSTRACT

A formulation for administering an extremely water-insoluble active agent is disclosed. More particularly, a self-emulsifying drug delivery system for extremely water-insoluble, lipophilic compounds is disclosed.

This application claims the benefit of U.S. Provisional Patent Application No. 60/220,376, filed on Jul. 24, 2000, the entire disclosure of which is herein incorporated by reference.

### BACKGROUND OF THE INVENTION

1. Field of the Invention

The invention relates to a formulation for extremely water-insoluble compounds. More particularly, the invention relates to a self-emulsifying drug delivery system for extremely water-insoluble, lipophilic drugs.

2. Description of Related Technology

In the pharmaceutical industry, a critical aspect of preparing a desirable product is the ability to properly formulate a poorly water-soluble drug, or active agent. Many drugs, including many indolinone analogs, are extremely insoluble in water and, as a result, the oral bioavailability of these drugs is low due to incomplete absorption. For example, some indolinone compounds have a solubility of only 10 nanograms per milliliter in water. Such solubility is generally believed to be too low for efficient oral absorption.

In addition, some formulations of extremely water-insoluble drugs, for example co-solvent based formulations, result in rapid precipitation of the drug upon aqueous dilution of the formulation under conditions simulating the gastrointestinal tract. Accordingly, scientists actively research and develop formulations for dissolving and solubilizing extremely water-insoluble compounds.

One method for administering extremely insoluble active agents is the self-emulsifying drug delivery system. A self-emulsifying drug delivery system is a uniphase liquid or semi-solid, typically comprising an oil and a surfactant, and having an oily nature, which forms an emulsion when contacted with an aqueous environment. Self-emulsifying drug delivery systems are easily administered and easy to manufacture. The self-emulsifying drug delivery systems offer the potential of improved oral absorption of active agents that are difficult to dissolve in aqueous solution.

Examples of extremely water-insoluble active agents are those compounds having a solubility in water of less than 100 micrograms per milliliter of water at room temperature. These extremely water-insoluble drugs can include various types of steroids, anticancer agents, antifungal agents, and antiinfective agents. It particularly would be beneficial to develop a self-emulsifying drug delivery system having suitable components for solubilizing and administering these extremely water-insoluble active agents in order to take advantage of the therapeutic activities of these compounds. Particularly, it would be beneficial to prepare a formulation for compounds in the indolinone class, which have demonstrated promising anticancer activity.

One possible component for a useful formulation is the hydrophilic, miscible polymer polyvinylpyrrolidone (“PVP”). Generally, polyvinylpyrrolidone is chemically compatible with a large variety of excipients. In the formulation art, however, polyvinylpyrrolidone, typically is used as a binder in tablet or pellet formulations. Primarily, the solid form of polyvinylpyrrolidone is incorporated as a dry powder into a blend of excipients to prepare tablet cores or pellets. For example, the literature reports using polyvinylpyrrolidone polymer dissolved in a solvent to improve the release rate of the active substance. See, for example, U.K. Patent No. 1,425,407, published Feb. 18, 1973. Typically, the solvent is evaporated to obtain a tablet formulation in its dry form. Examples of this use of polyvinylpyrrolidone are described in U.S. Pat. No. 5,776,495, issued Jul. 7, 1998; U.S. Pat. No. 6,027,747, issued Feb. 22, 2000; and International Publication No. WO 97/04749, published Feb. 13, 1997.

A less common use of polyvinylpyrrolidone involves suspending, stabilizing or increasing the viscosity of a topical or orally-administered suspension or solution, including emulsions. Examples of such use are described in European Patent Publication No. 214501 A2, published Mar. 18, 1987. When used as a suspending or stabilizing agent, the polyvinylpyrrolidone in the composition is present in small amounts, as determined by weight of the composition. Typically, the amount of polyvinylpyrrolidone in suspensions or emulsions ranges from less than about 1 wt. % to about 5 wt. % of the formulation. See, Handbook of Pharmaceutical Excipients, 2d edition, American Pharmaceutical Association, 1994, 392-399.

Polyvinylpyrrolidone also can be incorporated into a coating composition. Typically, in the context of a coating composition, polyvinylpyrrolidone is employed as a thickener. See, for example, International Publication No. WO 97/47285, published Dec. 18, 1997.

To date, no literature has been reported regarding the use of polyvinylpyrrolidone in an orally-administered self-emulsifying drug delivery system, particularly for aiding dissolution of an extremely water-insoluble drug. Moreover, only a limited body of literature reports using polyvinylpyrrolidone at concentrations beyond 5%, by weight. A beneficial formulation would solubilize a sufficient amount of an extremely water-insoluble active agent for therapeutic administration to an individual and would prevent precipitation of the drug under conditions simulated in the gastrointestinal tract.

### SUMMARY OF THE INVENTION

The invention provides a formulation for an extremely water-insoluble active agent. An extremely water-insoluble active agent typically has a solubility in water of less than about 100 micrograms per milliliter at room temperature. The active agent is incorporated in a suitable pharmaceutical vehicle. The vehicle comprises a polyvinylpyrrolidone polymer, a fatty acid, and a surfactant. When dispersed in an aqueous environment, the formulation spontaneously forms an emulsion wherein the active agent is partitioned and remains solubilized in the emulsified oil phase. The self-emulsifying formulation provides a useful dosage form for administering the active agent to provide enhanced bioavailability over conventional dosage forms.

The self-emulsifying formulation is useful for administering extremely water-insoluble active agents, such as active agents having anticancer activity. The formulation is particularly beneficial for administering lipophilic compounds, for example indolinone derivatives and other compounds which are extremely insoluble in water.

The above and other aspects, advantages, and novel features of the invention will become apparent from the following detailed description of the invention.

### DETAILED DESCRIPTION OF THE INVENTION

Therefore, in one aspect, the invention relates to a formulation for an extremely water-insoluble, lipophilic active agent in a vehicle, wherein the vehicle comprises polyvinylpyrrolidone, a fatty acid, and a surfactant. The formulation spontaneously forms an emulsion when dispersed in an aqueous environment. The extremely water-insoluble active agent typically has a solubility of less than 100 micrograms per milliter of water.

In another aspect, the invention relates to a method of preparing a self-emulsifying system containing an extremely water-insoluble active agent. The method comprises combining the extremely water-insoluble active agent with polyvinylpyrrolidone, either by solubilizing the active agent in polyvinylpyrrolidone directly, typically by first dissolving the polyvinylpyrrolidone in an organic solvent, or by dissolving the active agent in a solution of fatty acid and surfactant, which then is combined with a solution of polyvinylpyrrolidone dissolved in organic solvent.

In another aspect, the formulation can be used in a method to treat a patient in need of treatment with a steroidal, an antifungal, an antibacterial, or an anticancer medicament, by administering a composition comprising the extremely water-insoluble, lipophilic active agent in a vehicle comprising polyvinylpyrrolidone, a fatty acid, and a surfactant. In particular, the method can be used for cancer treatment, comprising the step of administering an anticancer active agent, such as indolinone compounds, in the formulation, either alone or in combination with additional medicament or formulations.

Use of the formulation comprising the extremely water-insoluble, lipophilic active agent in a vehicle comprising polyvinylpyrrolidone, a fatty acid, and a surfactant, for the manufacture of a medicament for therapeutic treatment, such as steroidal, antifungal, antibacterial, or anticancer treatment, also is contemplated herein.

The invention provides a formulation containing an extremely water-insoluble active agent in a pharmaceutically acceptable vehicle. The vehicle comprises (a) polyvinylpyrrolidone, (b) a fatty acid, and (c) a surfactant. The vehicle solubilizes the extremely water-insoluble drug in a liquid or semi-solid medium to achieve a high concentration. The improved dissolution and dispersion properties of this formulation affords improved bioavailability of the drug.

A particular advantage of the invention includes that the formulation provides high concentration of an extremely water-insoluble active agent. In addition, a self-emulsifying formulation of the invention reduces or eliminates precipitation of the active agent upon dilution of the formulation in simulated gastric fluid (pH 2, 0.01 N HCl). The self-emulsifying system can be easily encapsulated into gelatin capsules and administered orally into humans or mammals.

The incorporation of polyvinylpyrrolidone in the self-emulsifying hydrophobic formulation achieves a high concentration of an extremely water-insoluble active agent in the formulation. For this reason, the formulation is particularly suitable for the extremely water-insoluble active agents such as indolinone compounds, for example 3-[(2,4-dimethylpyrrol-5-yl)methylene]-2-indolinone and 3-[2,4-dimethyl-5-(2-oxo-1,2-dihydroindol-3-ylidenemethyl)-1H-pyrrol-3-yl]propionic acid. More particularly, the formulation is useful for the active agent, 3-[(2,4-dimethylpyrrol-5-yl)methylene]-2-indolinone, achieving a concentration of about 30 mg/g of active agent in the formulation. Upon contact with water, the invention generates a microemulsion that promotes excellent dispersion for rapid drug release at 37° C. and provides high oral bioavailability.

Polyvinylpyrrolidone is a synthetic polymer formed from linear 1-vinyl-2-pyrrolidinone groups. The use of polyvinylpyrrolidone has not been described previously as an excipient useful in self-emulsifying hydrophobic formulations. The degree of polymerization of the polymer affords polymers of various weights, by which the polyvinylpyrrolidone can be characterized. A polyvinylpyrrolidone useful in the present invention can have a molecular weight of about 2,500 to about 100,000. An increasing molecular weight of the polyvinylpyrrolidone polymer correlates to increasing viscosity, which is expressed as a K value. Polyvinylpyrrolidone polymers are commercially available from BASF Corporation (Parsippany, N.J., U.S.A.) under the trade name KOLLIDON™, and generally can be obtained in K values of 12, 15, 17, 25, 30, 60, and 90. The preferred polymers have a molecular weight of about 2,500 to about 20,000, which correspond to lower K values, such as K12 and K25. A sufficient amount of polyvinylpyrrolidone is used to dissolve the desired amount of the active agent. To achieve the full advantage of the present invention, the active agent is dissolved in the vehicle containing polyvinylpyrrolidone. The invention has a unique advantage in that the polyvinylpyrrolidone, which generally is used for preparing solid formulations, such as tablets or pellets, can dissolve an extremely water-insoluble, lipophilic active agent.

The preferred amount of polyvinylpyrrolidone in the formulation is about 5 wt. % to about 40 wt. % of the total formulation. A more preferred amount of polyvinylpyrrolidone in the formulation is about 10 wt. % to about 30 wt. %, and even more preferably about 10 wt. % to about 25 wt. %.

The polyvinylpyrrolidone can be dissolved in a pharmaceutically acceptable solvent to improve dissolution of the active agent. A suitable solvent typically is a pharmaceutically acceptable solvent, for example alcoholic solvents. Suitable solvents include, but are not limited to, ethanol, polyethylene glycol, propylene glycol, and mixtures thereof. The preferred solvent is ethanol.

The dissolution of the polyvinylpyrrolidone in the solvent generally is homogenous and sufficient to dissolve the desired amount of the drug. The amount of polyvinylpyrrolidone dissolved in the solvent generally is in the range of about 0.5 to about 3 parts of polyvinylpyrrolidone per one part of solvent. The amount of solvent preferably ranges from about 5 wt. % to about 30 wt. % based on the total weight of the formulation.

The fatty acid prevents or eliminates phase separation between the components of the formulation. Phase separation can occur when the water content of the formulation is above about 3%. The fatty acid comprises a linear or branched-chain hydrocarbon substituted with one or more carboxylic acid functional groups, and optionally with one or more hydroxy groups. Saturated and unsaturated fatty acids, preferably containing about 6 to about 22 carbon atoms, are suitable for the invention.

A preferred fatty acid is a linear, substantially unbranched fatty acid containing from about 6 to about 18 carbons. Examples of suitable fatty acids include, but are not limited to, hexanoic acid, octanoic acid, nonanoic acid, decanoic acid, lauric acid, linoleic acid, oleic acid, palmitic acid, and the like, or mixtures thereof.

The addition of a fatty acid improves the solubility and permits successful encapsulation of the formulation, typically into soft gelatin capsules (SGCs), hard gelatin capsules (HGCs), or hydroxypropyl methylcellulose (HPMC) capsules, at concentrations of about 30 mg/g of active agent. For 3-[(2,4-dimethylpyrrol-5-yl)methylene]-2-indolinone, the addition of the fatty acid allows preparation of a stable formulation even in the presence of about 5% to about 7% water, by weight of the total formulation, without phase separation or drug precipitation.

The amount of fatty acid preferably comprises about 5 wt. % to about 35 wt. % of the formulation. The formulation more preferably comprises about 5 wt. % to about 25 wt. % fatty acid. An even more preferable amount of the fatty acid is about 5 wt. % to about 15 wt. %.

The surfactant can be any suitable substance that generates emulsion droplets by dispersing the formulation in an aqueous environment. As used herein, the term “emulsion droplets” refers to microscopically dispersed droplets in an aqueous environment, generally having a droplet size of less than or equal to 50 μm, wherein each droplet comprises a surfactant layer surrounding an oil core.

A variety of pharmaceutically acceptable surfactants are suitable for use in the invention. Generally, surfactants suitable for the invention are nonionic surfactants, for example, polyoxylated castor oil, polyoxylated glycerides of fatty acid, polyethylene sorbitan fatty acid esters, polyglycolyzed glycerides, and the like, or mixtures thereof. Examples of surfactants useful for the invention include, polyoxyl 40 hydrogenated castor oil sold under the trade name, among the others, CREMOPHOR™ RH40 (BASF Corporation, Parsippany, N.J., U.S.A.); polyoxyl 35 castor oil sold under the trade name, CREMOPHOR™ EL or CREMOPHOR™ EL-P (both available from BASF Corporation); polyoxylated glycerol fatty acid esters sold under the trade name SOLUTOL™ HS-15, TAGA™ TO (Goldschmidt Chemical Corp. Hopewell, Va., U.S.A.), and PEGLICOL™ 6-oleate; polyoxyethylene sorbitan fatty acid esters; polyoxytheylene stearates; saturated polyglycolyzed glyerides; or poloxamers; all of which are commercially available. Polyoxyethylene sorbitan fatty acid esters can include polysorbates, for example, polysorbate 20, polysorbate 40, polysorbate 60, and polysorbate 80. Polyoxyethylene stearates can include polyoxyl 6 stearate, polyoxyl 8 stearate, polyoxyl 12 stearate and polyoxyl 20 stearate. Saturated polyglycolyzed glycerides are, for example, GELUCIRE™ 44/14 or GELUCIRE™ 50/13 (Gattefosse, Westwood, N.J., U.S.A.). Poloxamers used herein include poloxamer 124 and poloxamer 188. Each surfactant can be used individually or in combination with other suitable surfactants.

The surfactant generally comprises about 20 wt. % to about 70 wt. % of the total composition. More preferably, the formulation comprises about 30 wt. % to about 50 wt. % surfactant.

The addition of an antioxidant to the composition provides the beneficial advantage of increased shelf life to the product. Any antioxidant compatible with the formulation can be used. The preferred antioxidants retard oxidation of the active agent in the formulation to provide a stable, effective composition. Preferred antioxidants include, for example, ascorbic acid, ascorbyl palmitate, butylhydroxyanisole, butylhydroxytoluene, propyl gallate, sodium ascorbate, tocopherol, and the like, or mixtures thereof. An antioxidant is incorporated in a suitable amount to oxidize excess ions in the formulation. In a preferred formulation, the antioxidant comprises less than about 1 wt. % of the total formulation and, more preferably, from about 0.05 wt. % to about 0.5 wt. % of the total formulation.

If desired, the formulation may further include conventional pharmaceutical additives. Examples of pharmaceutical additives include, but are not limited to, co-surfactants (for examples, sodium lauryl sulfate), coloring agents, flavoring agents, preserving agents, stabilizers, and/or thickening agents.

The formulation can have a liquid or semi-solid form and, if desired, can be filled into a gelatin capsule. After administration, the capsule ruptures and releases the formulation. When the formulation contacts an aqueous environment, for example in the gastrointestinal tract, the formulation spontaneously forms an emulsion. One advantage of the invention is that active agents having poor water-solubility can be solubilized and formulated into a beneficial therapeutic formulation.

This benefit of the invention is best achieved with extremely water-insoluble active agents having a low solubility of less than 100 micrograms per milliliter of water. The extremely water-insoluble active agents having a log P equal or larger than 2 are considered lipophilic compounds, which are particularly suitable for the invention. The term “log P” refers to the logarithms of the partition coefficient of the drug between two immiscible phases, in this case, n-octanol and water. Examples of active agents suitable for the invention include, but are not limited to, active agents having steroidal, anticancer, antifungal, and antiinfective activity. Nonlimiting examples of compounds suitable for the invention are the extremely water-insoluble active agents, for example, progesterone, ketoconazole, itraconazole, metroxyprogesterone, and paclitaxel. Other compounds suitable for the invention are extremely water-insoluble indolinones. Preferred compounds for the formulation of the invention are disclosed in U.S. Pat. No. 5,792,783, issued Aug. 11, 1998, incorporated herein by reference, describing 3-heteroaryl-2-indolinone compounds of the formula:

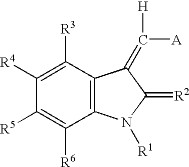

or a pharmaceutically acceptable salt, analog, or prodrug thereof, wherein:

R¹ is H or alkyl;

R² is O or S;

R³, R⁴, R⁵, and R⁶ are each independently selected from the group consisting of hydrogen, alkyl, alkoxy, aryl, aryloxy, alkaryl, alkaryloxy, halogen, trihalomethyl, S(O)R, SO₂NRR′, SO₃R, SR, NO₂, NRR′, OH, CN, C(O)R, OC(O)R, NHC(O)R, (CH₂)ₙCO₂R, and CONRR′;

A is a five membered heteroaryl ring selected from the group consisting of thiophene, pyrrole, pyrazole, imidazole, 1,2,3-triazole, 1,2,4-triazole, oxazole, isoxazole, thiazole, isothiazole, 2-sulfonylfuran, 4-alkylfuran, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-thiadiazole, 1,2,4-thiadiazole, 1,2,5-thiadiazole, 1,3,4-thiadiazole, 1,2,3,4-thiatriazole, 1,2,3,5-thiatriazole, and tetrazole, wherein said ring is optionally substituted at one or more positions with alkyl, alkoxy, aryl, aryloxy, alkaryl, alkaryloxy, halogen, trihalomethyl, S(O)R, SO₂NRR′, SO₃R, SR, NO₂, NRR′, OH, CN, C (O) R, OC (O) R, NHC(O)R, (CH₂)ₙCO₂R or CONRR′;

n is 0-3; and

R and R′ are, independently, H, alkyl or aryl.

As used herein, the term “pharmaceutically acceptable salt” refers to those salts which retain the biological effectiveness and properties of the free bases and which are obtained by reaction with inorganic acids, such as hydrochloric acid, hydrobromic acid, sulfuric acid, nitric acid, phosphoric acid, methanesulfonic acid, ethanesulfonic acid, p-toluenesulfonic acid, salicylic acid and the like, for example.

As used herein, the term “analogs” refers to a compound having the same basic structure as the parent compound, but with different atoms.

The term “prodrugs” as used herein refers to a derivative of an active agent that is converted into the parent compound in vivo. Prodrugs are often useful because, in some situations, they may be easier to administer than the parent drug. A prodrug may, for instance, be bioavailable by oral administration whereas the parent drug is not. The prodrug may also have improved solubility in pharmaceutical compositions over the parent drug. An example, without limitation, of a prodrug would be a compound, as defined above, that is administered as an ester.

The term “alkyl” refers to a straight-chain, branched, or cyclic saturated aliphatic hydrocarbon. Preferably, the alkyl group has 1 to 12 carbons. More preferably, the alkyl group is a lower alkyl having 1 to 7 carbons, more preferably 1 to 4 carbons. Typical alkyl groups include methyl, ethyl, propyl, isopropyl, butyl, isobutyl, tertiary butyl, pentyl, hexyl and the like. The alkyl group can be optionally substituted with one or more substituents selected from the group consisting of hydroxyl, cyano, alkoxy, O, S, NO₂, halogen, amino, and SH.

As used herein, the term “alkoxy” refers to an “—O-alkyl” group.

As used herein, the term “aryl” refers to an aromatic group which has at least one ring having a conjugated pi electron system and includes carbocyclic aryl, heterocyclic aryl and biaryl groups. The aryl group can be optionally substituted with one or more substituents selected from the group consisting of halogen, trihalomethyl, hydroxyl, SH, OH, NO₂, thioether, cyano (CN), alkoxy, alkyl, and amino.

As used herein, the term “aryloxy” refers to an “—O-aryl” group.

The term “alkaryl” as used herein refers to an alkyl that is covalently joined to an aryl group. Preferably, the alkyl is a lower alkyl.

As used herein, the term “alkylaryloxy” refers to an “—O-alkylaryl” group.

The term “carbocyclic aryl” as used herein refers to an aryl group wherein the ring atoms are carbon.

As used herein, the term “halogen” refers to a bromine, chlorine, fluorine, or iodine atom.

As used herein, the term “heterocyclic aryl” refers to an aryl group having from 1 to 3 heteroatoms as ring atoms, the remainder of the ring atoms being carbon. Heteroatoms include oxygen, sulfur, and nitrogen. Thus, heterocyclic aryl groups include furanyl, thienyl, pyridyl, pyrrolyl, N-lower alkylpyrrolo, pyrimidyl, pyrazinyl, imidazolyl, and the like.

As used herein, the term “amino” refers to a —N(R^{a})R^{b} group, wherein R^{a} and R^{b} are, independently, selected from the group consisting of hydrogen, alkyl, aryl, and alkylaryl.

The more preferred compounds for the formulation are those of formula (I) wherein the substituent A is a pyrrole group optionally substituted with a substituent selected from the group consisting of alkyl, alkoxy, halogen, and —COR, wherein R is as previously defined. Yet more preferred compounds for the formulation are 3-[(2,4-dimethylpyrrol-5-yl)methylene]-2-indolinone, 3-[2,4-dimethyl-5-(2-oxo-1,2-dihydroindol-3-ylidenemethyl)-1H-pyrrol-3-yl]propionic acid, and analogs, prodrugs, and salts thereof. The preferred amount of the active agent is from about 1 wt. % to about 4 wt. % of the formulation.

The formulation allows for the oral administration of extremely water-insoluble active agents to achieve sufficiently high oral bioavailability to treat a disease, condition, or symptom of a disease. The improved formulation is achieved by solubilizing the extremely water-insoluble compound in a solution of polyvinylpyrrolidone dissolved in pharmaceutically acceptable organic solvent, preferably ethanol. The resulting polyvinylpyrrolidone solution is incorporated into a mixture comprising the fatty acid and the surfactant.

The fatty acid and surfactant are useful excipients for providing a self-emulsifying formulation, which spontaneously forms an emulsion upon contact with an aqueous environment. Generally, conventional usage dictates that polyvinylpyrrolidone is a component of solid, tablet or pellet formulations. The invention provides a beneficial formulation by incorporating the advantages of polyvinylpyrrolidone into a self-emulsifying formulation.

In a formulation of the invention, the solubility of extremely water-insoluble, lipophilic active agents, for example 3-[(2,4-dimethylpyrrol-5-yl)methylene]-2-indolinone can be increased from about 15 mg/g of drug dissolved in a vehicle free of polyvinylpyrrolidone to over 30 mg/g in a vehicle of the present invention, to provide a sufficiently high bioavailability for therapeutic treatment. Although it is particularly difficult to solubilize, 3-[(2,4-dimethylpyrrol-5-yl)methylene]-2-indolinone has demonstrated promising anticancer activity. In a typical aqueous formulation, the solubility of the compound is limited to approximately 10 nanograms per milliliter at room temperature. A benefit of the invention is to prepare a formulation of higher drug concentration for extremely water-insoluble compounds, at concentrations of ˜30 mg/ml for 3-[(2,4-dimethylpyrrol-5-yl)methylene]-2-indolinone.

The formulation can be achieved by combining the active agent, polyvinylpyrrolidone, fatty acid, surfactant, and alcoholic solvent to obtain a homogenous formulation. For example, the formulation is prepared by dissolving the active agent in a premixed solution of fatty acid and surfactant, then blending the solution obtained therefrom with a premixed solution of polyvinylpyrrolidone dissolved in alcoholic solvent. The formulation then is stirred until homogeneous. In another example, the active agent can be dissolved in the polyvinylpyrrolidone and stirred with a premixed solution of fatty acid and surfactant until homogenous.

The formulation can be filled into an HPMC capsule or a gelatin capsule, including hard- and soft-shell gelatin capsules. Typically, the gelatin capsule comprises gelatin with an optional amount of plasticizer and other optional excipients. Examples of other excipients include, but are not limited to, dyes, colorants, preservatives, and the like.

Preferably, the formulation contains about 1 part to about 2 parts by weight of fatty acid per 1 part to about 3 parts of polyvinylpyrrolidone. The amount of surfactant in the formulation relative to the polyvinylpyrrolidone ranges from about 1 to about 10 parts by weight per part of PVP. Preferably, about 0.5 to about 3 parts by weight of polyvinylpyrrolidone dissolves in one part by weight of ethanol. The amount of polyvinylpyrrolidone in the formulation is sufficient to dissolve the desired active agent. A preferred formulation wherein the fatty acid and polyvinylpyrrolidone are in a weight ratio of about 2:1 to about 1:3 (fatty acid: polyvinylpyrrolidone) and the surfactant and polyvinylpyrrolidone are in a weight ratio of about 10:1 to about 1:1 (surfactant: polyvinylpyrrolidone) provides an oily liquid that, when mixed with sufficient amount of aqueous medium, forms an emulsion of the active agent in oily droplets.

The gelatin capsules typically can be administered orally. The formulation also can be in the form of a liquid or semi-solid solution for oral, parenteral, rectal, or topical application. The preferred dosage form is a liquid contained in a soft-shell gelatin capsule or hard gelatin capsule. The daily dosage and therapeutic regimen of administering the formulation can be determined by one with skill in the art of treating and preventing medical conditions. To provide guidance regarding the use of the invention with respect to the treatment of cancer, the formulation can be administered in an amount from about 0.01 to about 200 milligrams of active agent per square meter of surface area to be treated. However, such amount should not entirely be limited by the description herein. Any useful amount of the active agent can be incorporated into the formulation.

When the formulation incorporates an anticancer active agent, the formulation can be used in a method of treating and/or preventing cancer in a patient. The preferred anticancer agent for use in the formulation and method of treating and preventing cancer is an indolinone compound, preferably 3-[(2,4-dimethylpyrrol-5-yl)methylene]-2-indolinone or 3-[2,4-dimethyl-5-(2-oxo-1,2-dihydroindol-3-ylidenemethyl)-1H-pyrrol-3-yl]propionic acid. The active agent can be used either alone or co-administered with additional active agents. Examples of active agents suitable for co-administration with a formulation of the present invention include, but are not limited to, vascular endothelial growth factor (VEGF), 5-fluorouracil (5-FU), leucovorin, CAMPTOSAR™ (irinotecan HCl), epirubicin, taxotere, taxol, carboplatin, gemcitabine, cisplatin, oxaliplatin, 5-azacitidine, and other signal transduction inhibitors, such as HERCEPTIN® (trastazumab) and IRESSA® (inhibitor of epidermal growth factor receptor tyrosine kinase (EGFR-TK)), as well as other cytostatics, for example matrix metalloproteinase inhibitors (MMPIs), avB3 inhibitors, FITs, and the like. Moreover, it is possible that additional active agents, particularly anticancer active agents, having suitable properties, for example having similar solubility, can be incorporated into the vehicle of the invention.

The invention can be better understood in the context of the following examples, which are meant to provide an illustration of, and are not limiting of the invention in any way. Without further elaboration, it is believed that one skilled in the art can, using the preceding description, practice the present invention to its fullest extent.

### EXAMPLE 1

### Determination of Oral Bioavailability

Formulations A-F, shown below, are described in Table 1, which summarizes the composition of each formulation tested.

TABLE 1
Vehicle Composition of Test Formulations

| Vehicle | Amount (mg/g) | | | | | |
|---|---|---|---|---|---|---|
| Composition¹ | A | B | C | D | E | F |
| 100% ethanol | 66 | 60 | 80 | 80 | 60 | — |
| PEG-600 | — | — | — | — | — | 100 |
| PVP (PK 12) | 134 | 120 | 160 | 240 | 180 | — |
| CREMOPHOR ™ EL | 300 | 475 | 100 | — | 160 | 100 |
| CREMOPHOR ™ RH40 | 400 | — | — | 460 | — | — |
| GELUCIRE ™ 44/14 | — | — | 500 | — | 470 | 700 |
| GDO/GMO (8:2) | 100 | — | — | — | — | 100 |
| Oleic Acid | — | — | 150 | — | 120 | — |
| Octanoic Acid | — | 200 | — | — | — | — |
| CAPMUL ™ MCM | — | 145 | — | 216 | — | — |
| MIGLYOL ™ 812 | — | — | — | — | — | 100 |
| Tocopherol | — | — | — | 22 | 55 | — |
| Ascorbyl | — | — | — | 22 | 55 | — |
| Palmitate | | | | | | |
| ¹The abbreviations and trade names used herein denote the following: PEG-600 refers to polyethylene glycol having an average of 600 moles of ethylene oxide; PVP (PK 12) refers to polyvinylpyrrolidone having a K value of 12; GDO and GMO refer to glycerol dioleate and glycerol monooleate, respectively, CAPMUL ™ MCM (Abitech, Columbus, Ohio, U.S.A.) is the trade name for a mixture of monoglycerides of caprylic and capric | | | | | | |
| # acids and MIGLYOL ™ 812 (Hüls America, Piscataway, New Jersey, U.S.A.) is a mixed triester of glycerin with caprylic, capric, and stearic acids. The weight percentage is based on the total weight of the composition. | | | | | | |

To prepare the formulations above, 30 mg of 3-[(2,4-dimethylpyrrol-5-yl)methylene]-2-indolinone was dissolved in ethanol, polyethylene glycol, or a mixture thereof, in the amounts, by weight, above. The dissolved active agent was combined with the remaining components of the formulation to obtain formulations A-F, respectively. Three additional formulations were prepared for comparison: micronized active agent in the vehicle of formulation E (150 mg/g); micronized active agent suspended in a mixture of GELUCIRE® 44/14 and lecithin (150 mg/g); and a 10%, by weight, solution of the active agent in lactose.

Drug concentrations in the blood of the test rats were plotted against the time after the drug is administered through an intravenous (i.v.) or oral route. The AUCs (the Area Under the Plasma Concentration-Time Curve) were recorded and integrated using the trapezoidal rule to calculate the absolute bioavailability as shown in Table 2 below.

$$Absolute bioavailability ( \% ) = \frac{{( AUC )}_{oral} / {Dose}_{oral}}{{( AUC )}_{iv} / {Dose}_{iv}}$$

Male beagle dogs were also selected for the in vivo oral bioavailability study. Each dog in the weight range of 11.5 kg-17.5 kg was fasted overnight prior to dosing. Each formulation was orally administered to a group of dogs (n=4) at a 10 mg/kg dose. The formulation of high concentration of the active agent, 3-[(2,4-dimethylpyrrol-5-yl)methylene]-2-indolinone (˜30 mg/g), was encapsulated in gelatin capsules and administered. Serial blood samples of 2 mL were obtained from the jugular vein at 20 and 40 minutes and 1, 2, 4, 6, 8, 12, and 24 hours after dosing. These blood samples were analyzed using a HPLC assay specific for the compound. The blood concentrations of the compound are plotted against the time and the AUCs were obtained to calculate the absolute bioavailability. The results are reported below in Table 2. The designations A-F in Table 2 denote the formulations as described above in Table 1.

TABLE 2
Comparison of Pharmacokinetics for
Various Dosage Forms

| | | | Absolute Oral |
|---|---|---|---|
| | Dose | AUC | Bioavailability |
| Formulation | (mg/kg) | (nM · hr) | (%) |
| A (30 mg/g) | 10 | 1904 ± 2134 | 10 ± 12 |
| B (30 mg/g) | 10 | 2549 ± 2169 | 13 ± 13 |
| C (30 mg/g) | 10 | 2723 ± 1714 | 15 ± 12 |
| D (30 mg/g) | 10 | 2311 ± 2011 | 12 ± 10 |
| F (30 mg/g) | 10 | 1243 ± 1921 | 6 ± 8 |
| Micronized bulk | 10 | 228 ± 340 | 1 ± 1 |
| drug in vehicle E | | | |
| Micronized bulk | 10 | 47 ± 64 | 0.2 ± 0.3 |
| drug suspension | | | |
| (150 mg/g) in | | | |
| GELUCIRE ™ and | | | |
| Lecithin | | | |
| 10% bulk drug in | 50 | 0 | 0 |
| lactose | | | |

As shown in Tables 1 and 2, the self-emulsifying drug delivery systems containing polyvinylpyrrolidone achieved 10% to 15% oral bioavailability of 3-[(2,4-dimethylpyrrol -5-yl)methylene]-2-indolinone. In contrast, the tablet and oil suspension formulations show that the conventional formulations only achieve 0% to 1% oral bioavailability of 3-[(2,4-dimethylpyrrol-5-yl)methylene]-2-indolinone.

### EXAMPLE 2

### General Methods for Preparing a Self-Emulsifying Drug Delivery System for an Extremely Water-Insoluble Drug

A mixture of polyvinylpyrrolidone arid ethanol was prepared for use in the formulation according to the following steps:

1) Weigh the polyvinylpyrrolidone in a glass flask containing a stir bar, then add the required amount of ethanol (EtOH) into the flask with hand mixing.

2) Cap the flask and heat the flask in a 60° C. water bath. Stir the PVP/EtOH solution in the flask until the mixture is homogeneous.

3) Cool the flask to room temperature.

The self-emulsifying formulation was prepared according to the following steps below:

4) Weigh the amount of the excipients listed below into a flask containing a stir bar in the following order:

ascorbyl palmitate;

tocopherol;

oleic acid;

CAPMUL® MCM;

CREMOPHOR® RH40;

Then cap the flask.

5) Heat the flask in a 65-70° C. water bath. Stir the solution in the flask until the mixture is homogeneous.

6) Add the amount of active agent and cap the flask. Repeat step 5, above, until the mixture is homogeneous.

7) Add the PVP/EtOH pre-prepared mixture and cap the flask. Repeat step 5.

The following formulations G-J were prepared with 3-[(2,4-dimethylpyrrol-5-yl)methylene]-2-indolinone active agent according to the methods above.

TABLE 3
Examples of Vehicle Compositions Containing
3- [(2, 4-Dimethylpyrrol-5-yl)methvlene] -2-indolinone

| Vehicle | Amount of Components (mg/g) | | | |
|---|---|---|---|---|
| Composition | G | H | I | J |
| Active agent | 30 mg/g | 30 mg/g | 30 mg/g | 30 mg/g |
| PVP (PK 12) | 210 | 150 | 205 | 205 |
| 100% ethanol | 70 | 50 | 65 | 65 |
| CREMOPHOR ™ EL | — | — | 110 | 130 |
| CREMOPHOR ™ RH40 | 460 | 460 | — | — |
| GELUCIRE ™ 44/14 | — | — | 480 | 460 |
| CAPMUL ™ MCM | — | 200 | — | — |
| GDO/GMO (8:2) | — | — | — | 100 |
| Oleic Acid | 120 | — | 100 | — |
| Octanoic Acid | — | 100 | — | — |
| Tocopherol | 5 | 5 | 5 | 5 |
| Ascorbyl | 5 | 5 | 5 | 5 |
| Palmitate | | | | |

The invention is not to be limited in scope by the exemplified embodiments that are intended as illustrations of single aspects of the invention. Various modifications of the invention in addition to those described herein will be apparent to those skilled in the art from the foregoing description. Such modifications are intended to fall within the scope of the appended claims.

What is claimed is:

1. A self-emulsifying drug delivery system comprising a mixture of an extremely water-insoluble, lipophilic active agent; polyvinylpyrrolidone; a fatty acid; and a surfactant.

2. The self-emulsifying drug delivery system of claim 1, wherein the weight ratio of said fatty acid to said polyvinylpyrrolidone is about 2:1 to about 1:3, and the weight ratio of said surfactant to said polyvinylpyrrolidone is about 10:1 to about 1:1.

3. The self-emulsifying drug delivery system of claim 1, wherein the extremely water-insoluble, lipophilic active agent has a log P equal or greater than 2, and the extremely water-insoluble, lipophilic active agent has a solubility of less than 100 micrograms per milliliter of water.

4. The self-emulsifying drug delivery system of claim 1, wherein the polyvinylpyrrolidone has a molecular weight of about 2,500 to about 100,000.

5. The self-emulsifying drug delivery system of claim 4, wherein the polyvinylpyrrolidone has a molecular weight of about 2,500 to about 20,000.

6. The self-emulsifying drug delivery system of claim 1, wherein the amount of polyvinylpyrrolidone is about 5% to about 40%, by weight of the self-emulsifying drug delivery system.

7. The self-emulsifying drug delivery system of claim 1, wherein the amount of fatty acid is about 5% to about 35%, by weight of the self-emulsifying drug delivery system.

8. The self-emulsifying drug delivery system of claim 1, wherein the amount of fatty acid is about 5% to about 15%, by weight of the self-emulsifying drug delivery system.

9. The self-emulsifying drug delivery system of claim 1, wherein the fatty acid is a fatty acid containing from about 6 to about 18 carbons.

10. The self-emulsifying drug delivery system of claim 9, wherein the fatty acid is selected from the group consisting of hexanoic acid, octanoic acid, nonanoic acid, decanoic acid, lauric acid, linoleic acid, oleic acid, palmitic acid, and mixtures thereof.

11. The self-emulsifying drug delivery system of claim 1, wherein the surfactant is selected from the group consisting of polyoxylated castor oil, polyoxylated glycerides of fatty acids, polyoxyethylene sorbitan fatty acid esters, polyglycolyzed glycerides, and mixtures thereof.

12. The self-emulsifying drug delivery system of claim 1, wherein the surfactant is selected from the group consisting of polyoxyl 35 castor oil and polysorbate 80.

13. The self-emulsifying drug delivery system of claim 1, wherein the amount of surfactant is about 20% to about 70%, by weight of the self-emulsifying system.

14. The self-emulsifying drug delivery system of claim 13, wherein the amount of the surfactant is about 30% to 50%, by weight of the self-emulsifying system.

15. The self-emulsifying drug delivery system of claim 1, further comprising an antioxidant selected from the group consisting of ascorbic acid, ascorbyl palmitate, butylhydroxyanisole, butylhydroxytoluene, propyl gallate, sodium ascorbate, tocopherol, and mixtures thereof.

16. The self-emulsifying drug delivery system of claim 1, further comprising a pharmaceutically acceptable organic solvent.

17. The self-emulsifying drug delivery system of claim 14, wherein the solvent is selected from the group consisting of ethanol, a polyethylene glycol, propylene glycol, and mixtures thereof.

18. The formulation of claim 1, comprising:
about 1 wt. % to about 4 wt. % said active agent;
about 5 wt. % to about 40 wt. % said polyvinylpyrrolidone;
about 5 wt. % to about 35 wt. % said fatty acid; and
about 20 wt. % to about 70 wt. % said surfactant.

19. The formulation of claim 1, wherein the active agent is a steroid, an anticancer agent, an antifungal agent, or antiinfective agent.

20. The formulation of claim 1, wherein the active agent is selected from the group consisting of progesterone, ketoconzaole, itraconazole, metroxyprogesterone, and paclitaxel.

21. The formulation of claim 1, wherein the active agent is a compound of the formula: 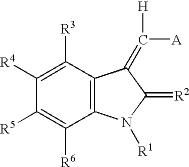
or a pharmaceutically acceptable salt, analog, or prodrug thereof, wherein:
R¹ is H or alkyl;
R² is O or S;
R³, R⁴, R⁵, and R⁶ are each independently selected from the group consisting of hydrogen, alkyl, alkoxy, aryl, aryloxy, alkaryl, alkaryloxy, halogen, trihalomethyl, S(O)R, SO₂NRR′, SO₃R, SR, NO₂, NRR′, OH, CN, C(O)R, OC(O)R, NHC (O)R, (CH₂ₙCO₂R, and CONRR′;
A is a five membered heteroaryl ring selected from the group consisting of thiophene, pyrrole, pyrazole, imidazole, 1,2,3-triazole, 1,2,4-triazole, oxazole, isoxazole, thiazole, isothiazole, 2-sulfonylfuran, 4-alkylfuran, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-thiadiazole, 1,2,4-thiadiazole, 1,2,5-thiadiazole, 1,3,4-thiadiazole, 1,2,3,4-thiatriazole, 1,2,3,5-thiatriazole, and tetrazole, wherein said ring is optionally substituted at one or more positions with alkyl, alkoxy, aryl, aryloxy, alkaryl, alkaryloxy, halogen, trihalomethyl, S(O)R, SO₂NRR′, SO₃R, SR, NO₂, NRR′, OH, CN, C(O)R, OC(O)R, NHC(O)R, (CH₂)ₙCO₂R or CONRR′;
n is 0-3; and
R and R′ are each independently H, alkyl or aryl.

22. The formulation of claim 21, wherein the active agent is a compound of formula (I) wherein A is pyrrole optionally substituted with a substituent selected from the group consisting of alkyl, alkoxy, halogen, and —COR.

23. The formulation of claim 21, wherein the active agent is 3-[(2,4-dimethylpyrrol-5-yl)methylene]-2-indolinone or a salt, analog, or prodrug thereof.

24. The formulation of claim 21, wherein the active agent is 3-[2,4-dimethyl-5-(2-oxo-1,2-dihydroindol-3-ylidenemethyl)-1H-pyrrol-3-yl]propionic acid or a salt, analog, or prodrug thereof.

25. The formulation of claim 1, wherein the formulation is filled into a gelatin capsule.

26. The formulation of claim 25, wherein the gelatin capsule is a hard-shelled gelatin capsule, a soft-shelled gelatin capsule, or a hydroxypropyl methylcellulose capsule.

27. The formulation of claim 1, wherein the formulation is administered orally, parenterally, rectally, or topically.

28. A method of treating and/or preventing a condition in need of a therapeutic regimen comprising a steroid, an antifungal agent, an antibacterial agent, or an anticancer agent, the method comprising the step of administering a self-emulsifying system comprising a mixture of a therapeutically effective amount of at least one extremely water-insoluble, lipophilic active agent; polyvinylpyrrolidone; a fatty acid; and a surfactant to an individual in need thereof.

29. The method of claim 28, wherein the weight ratio of said fatty acid to said polyvinylpyrrolidone is about 2:1 to about 1:3 and the weight ratio of said surfactant to said polyvinylpyrrolidone is about 10:1 to about 1:1.

30. The method of claim 28, wherein the extremely water-insoluble, lipophilic active agent has a log P of equal or greater than 2, and the extremely water-insoluble, lipophilic anticancer active agent has a solubility of less than 100 micrograms per milliliter of water.

31. The method of claim 28, wherein the extremely water-insoluble, lipophilic active agent is an anticancer agent selected from the group consisting of paclitaxel or an indolinone compound.

32. The method of claim 28, wherein the formulation is administered in combination with at least one additional active agent.

33. The method of claim 32, wherein the formulation is administered in combination with an active agent selected from the group consisting of vascular endothelial growth factor, 5-fluorouracil, leucovorin, irinotecan HCl, epirubicin, taxotere, taxol, carboplatin, gemcitabine, cisplatin, oxaliplatin, 5-azacitidine, a signal transduction inhibitors, a cytostatic compound, and mixtures thereof.

34. The method of claim 28, wherein the extremely water-insoluble, lipophilic active agent is a steroid, an antifungal agent, or antibacterial agent selected from the group consisting of progesterone, ketoconazole, itrazole, and metroxyprogesterone.

35. Use of a composition comprising an extremely water-insoluble, lipophilic active agent, polyvinylpyrrolidone, a fatty acid, and a surfactant, for the manufacture of a medicament for a condition in need of a therapeutic regimen comprising an active agent selected from the group consisting of a steroid, an antifungal agent, an antibacterial agent, and an anticancer agent.
